(19) United States
(12) Patent Application Publication
(10) Pub. No.: US 2006/0165616 A1
(43) Pub. Date: Jul. 27, 2006

(54) Microemulsion containing anti-uv filters and/or anti-dandruff agents

(76) Inventors: Michael Brock, Schermbeck (DE); Thorsten Hanning, Dulmen (DE); Eva-Maria Koberstein, Recklinghausen (DE); Heinz Napierala, Herten (DE)

Correspondence Address: C. James Bushman;Browning Bushman, Suite 1800, 5718 Westheimer,, Houston, TX, 77057-5771, US

(21) Appl. No.: 10/523,243

(22) PCT Filed: Jul. 22, 2003
(86) PCT No.: PCT/DE03/02454

§ 371 Date: Oct. 12, 2005

(30) Foreign Application Priority Data
Jul. 22, 2002 (DE) 102 33 330.0

(51) Int. Cl. A61K 8/46

(52) U.S. Cl. 424/59

(57) ABSTRACT

This invention relates to microemulsions containing alkanolammonium salts of alkylsulfates and/or alkylpolyalkyleneglycolethersulfates, UV filters and/or antidandruff substances. The invention also relates to the use of said emulsions for cosmetic and/or medicinal-dermatologic applications.

The present invention relates to microemulsions containing UV filters and/or antidandruff substances and to their uses.

When exposed to sunlight, normal skin gets pigmented due to the formation of melanin. UV-A light makes the melanin in the epidermis dark, while UV-13 rays account for the formation of melanin. When the skin is exposed for a long time to intensive sunlight, the pigmentation lags behind as a result of the abundant presence of UV-B rays. This may cause redness or inflammation of the skin (erythema or sunburn) and may even produce blisters from burns. A large variety of sunscreens are available in order to at least mitigate these undesirable effects. These products are mainly creams, lotions, or sprays, which are applied to the skin shortly before or during the exposure to intensive sunlight. The products are mostly milky-turbid macroemulsions, which primarily contain UV filters besides some regenerating agents.

High-selling sunscreen products do not fulfill the consumers' request for multifunctional cosmetic products; The consumers demand cosmetic products performing several functions, e.g. cleaning and treatment of the skin, with just one application. They also want transparent products, which meet the increasing requirement for esthetically acceptable formulations. Hence, there is a demand for preparations, which are capable of cleaning the skin, repairing it with therapeutic substances, and providing sun protection in one application.

Liquid compositions which are intended for use both as body cleaners and care preparations need to fulfill different requirements, e.g. combining the cleaning properties of an aqueous surfactant formulation with the cosmetic properties of an oil component. Skin and hair are usually cleaned with surfactants, which effect more or less pronounced swelling and subsequent dehydration of the stratum corneum of the skin, thereby impairing the protective mechanism of the skin surface. Therefore, skincare components allowing regeneration of the skin are increasingly added to customary skin-cleaning preparations. The type of oil component, the amount used in a formulation, the percentage of the aqueous phase and its composition are frequently predetermined by the requirements of the individual applications. Marketable compositions, which meet said requirements, are usually macroemulsions. Same are turbid and thermodynamically instable, i.e. they separate irreversibly after a while. Alternative products are microemulsions, which are esthetically favorable, optically transparent, thermodynamically stable, and thus storable.

The expert has no difficulties in finding a suitable surfactant or combination of surfactants available on the market for producing a macroemulsion. The making of a microemulsion, however, presents some problems because the phase spaces of a macroemulsion of an oil-water-surfactant mixture are significantly larger than. Those of microemulsions. Moreover, depending on the application, further functional auxiliaries may be required to be incorporated the microemulsion without impairing its stability. In many cases it is also desirable to incorporate both water-soluble and oil-soluble components without making the microemulsion instable.

Customary functional auxiliaries, which are desirable in microemulsions, can, for example, be UV filters in the production of cleansing and grooming preparations comprising sunscreens and antidandruff substances, which are appropriate for use in antidandruff hair shampoos.

It is the object of the present invention to provide cosmetic and medicinal-dermatologic microemulsions in which both oil-soluble and water-soluble light protection filters or antidandruff substances can be incorporated without impairing the stability of the microemulsion and which are particularly suitable for cleaning and treating the skin.

According to the present invention, the problem has been resolved by microemulsions containing at least the following components:
(A) 0.5 to 70% by weight of alkanolammonium salts of alkylsulfates and/or alkylpolyalkyleneglycolethersulfates having the following structure
   R¹—O—(CₚH₂ₚO)ₘ—SO₃—HN⁺R²R³R⁴,
   wherein
   R¹=is a C₈- to C₂₀-hydrocarbon radical,
   p=is an integer from 2 to 5, where p may be different for each m,
   R²=is H, a C₁- to C₆-alkyl or a C₂- to C₄-hydroxyalkyl, particularly hydroxyisopropyl,
   R³=is H, a C₁- to C₆-alkyl or a C₂- to C₄-hydroxyalkyl, particularly hydroxyisopropyl,
   R⁴=is a C₂- to C₄-hydroxyalkyl, particularly hydroxyisopropyl, and
   m=is an integer from 0 to 7,
   or mixtures thereof,
(B) 20 to 95% by weight of water,
(C) 0.1 to 20% by weight of one or more oil component(s), and
(D) 0.1 to 20% by weight of one or more mono- or polyhydric C2- to C24-alcohol(s), and
(E.1) 0.1 to 15% by weight of one or more UV filter(s) and/or
(E.2) 0.1 to 3% by weight of one or more antidandruff substance(s),
   each percentage hereof based on the total composition.

Preferred embodiments of the aforementioned composition are set forth hereinbelow or in the subordinate claims.

Microemulsions containing alkanolammonium salts of alkylsulfates and/or alkylpolyalkyleneglycolethersulfates have been disclosed in WO 00/47166-A2 and are explicitly incorporated herein by reference as part of the disclosure of this application.

Moreover, the microemulsions of the subject invention may optionally contain independently of one another at least one of the following components:
(F) greater than 0 to 20% by weight, preferably 3 to 15% by weight of one or more additional surfactant(s),
(G) greater than 0 to 20% by weight, preferably 1 to 15% by weight, or 2 to 10% by weight of one or more electrolyte(s), and/or
(H) greater than 0 to 10% by weight, preferably 0.1 to 8% by weight of one or more additive(s).

Most advantageously, the microemulsions contain independently of one another the abovementioned components in the quantities set forth hereinbelow:
(A) 2 to 60% by weight, preferably 5 to 40% by weight,
(B) 30 to 80% by weight, preferably 40 to 60% by weight,
(C) 0.5 to 15% by weight, preferably 1 to 10% by weight,
(D) 0.1 to 9% by weight, preferably 0.5 to 9% by weight,
(E.1) 0.5 to 10% by weight, preferably 1 to 8% by weight and/or
(E.2) 0.2 to 2% by weight, preferably 0.3 to 1.5% by weight, and optionally
(F) greater a 0 to 20% by weight, preferably 3 to 15% by weight of additional surfactants,
(G) greater than 0 to 20% by weight, preferably 1 to 12% by weight of electrolytes, and/or
(H) greater than 0 to 10% by weight, preferably 0.1 to 8% by weight of additives,
   wherein furthermore most advantageously
(F) is or contains as an additional surfactant a triglyceride alkoxylated with ethyleneoxide and/or propyleneoxide and subsequently esterified, wholly or in part, with C₆- to C₂₂-fatty acids, preferably in quantities from 1 to 20% by weight.

Contrary to macroemulsions, the microemulsions of the present invention are thermodynamically stable, optically transparent, macroscopically homogeneous mixtures of two immiscible liquids, namely, water (B) and an oil component (C) to which the surfactant molecules mentioned under (A), supra, were added. The microemulsions of the invention can be prepared, for example, at temperatures ranging from 15 to 80° C., preferably below 55° C. They are stable at least up to 60° C. The average particle size of the disperse phase is preferably less than 100 nm.

The microemulsions as claimed herein normally do not form mesomorphic (liquid crystal) phases within a wide range of compositions. They are most suitable for cosmetic and/or medicinal-dermatologic applications. In particular, they are employed as or in body cleaners and body care preparations.

The aforementioned compositions of the present invention most preferably contain alkanolammonium salts of the alkylsulfates and/or alkylpolyalkyleneglycolethersulfates of the abovementioned general structure. Preferably, they have independently of one another the following residues:
R¹=C₁₂- to C₁₆-alkyl, the allyl residue being linear and saturated,
p=2 or 3, where p can be different for each m,
R²=H or hydroxyisopropyl,
R³=H or hydroxyisopropyl,
R⁴=hydroxyisopropyl, and
m=0, 1 or 2.

Favorable embodiments of the present invention with respect to the components (C) to (H) are set out below.

Oil Component (C)

The oil components of the present invention are advantageously chosen from the group of lecithins and the group of mono-, di-, and/or triglycerides of saturated and/or unsaturated, branched and/or linear alkylcarboxylic acids having a chain length from 8 to 24 carbon atoms, particularly 12 to 18. The fatty acid triglycerides can advantageously be synthetic, semisynthetic, or natural oils, such as soya oil, castor oil, olive oil, safflower oil, wheatgerm oil, grapeseed oil, sunflower oil, peanut oil, almond oil, palm oil, coconut oil, thistle oil, evening primrose oil, rape oil, etc.

The oil component can furthermore comprise Vaseline, paraffin oil, and polyolefins. Moreover, the oil components according to the present invention can advantageously be chosen from the group of esters of saturated and/or unsaturated, branched and/or linear alkylcarboxylic acids having a chain length from 3 to 30 carbon atoms and of saturated and/or unsaturated, branched and/or linear alcohols having a chain length from 3 to 30 carbon atoms. It is furthermore advantageous to choose the oil components from the group of esters of aromatic carboxylic acids and saturated and/or unsaturated, branched and/or linear alcohols having a chain length from 3 to 30 carbon atoms, which ester oils can advantageously be chosen from the group of isopropyl myristate, isopropyl palmitate, isopropyl stearate, isopropyl oleate, n-butyl stearate, n-hexyl laurate, n-decyl oleate, isooctylstearate, isononylstearate, isononylisononanoate, 2-ethylhexylpalmitate, 2-ethylhexyllaurate, 2-hexyldecyl-stearate, 2-octyldodecylpalmitate, oleyl oleate, oleyl erucate, erucyl oleate, erucyl erucate, and synthetic, semisynthetic, and natural mixtures of such esters, e.g. jojoba oil.

Furthermore, the oil component can advantageously be chosen from the group of branched and linear hydrocarbons and hydrocarbon waxes and silicone oils. Any mixtures of the aforesaid oil components are also advantageous within the meaning of the present invention.

Alcohols (D)

The microemulsions claimed herein contain mono- or polyhydroxy, preferably mono-, di-, or trihydroxy C₂- to C₂₄-alcohols, preferably saturated and/or branched and/or linear alcohols. Examples of such alcohols include ethanol, propanol, isopropyl alcohol, butanol, pentanol, hexanol, heptanol, octanol, 2-ethylhexanol, lauryl alcohol, myristol alcohol, palmityl alcohol, steryl alcohol, oleyl alcohol, elaidyl alcohol, guerbet alcohols, and alkylene glycols such as ethylene glycol, propylene glycol, and glycerol. Propylene glycol is particularly preferred.

UV Filters (E.1)

It has surprisingly been discovered that both water-soluble and oil-soluble UV filters can reliably be incorporated into the microemulsions.

Microemulsions containing UV filters can favorably be employed for cleaning and treating the skin. Once applied, the UV filter remains on the skin and serves as a sunscreen. It is thus no longer necessary to reapply sun cream after washing the skin.

By the term ‘UV filter’ as used herein is meant any organic substance which is capable of absorbing ultraviolet rays and converting the absorbed energy into radiation of longer wavelengths, e.g. heat. A distinction is made between UVA filters and UVB filters, depending on the range of radiation that is absorted. UVA filters filter long-wave UVA rays (320 to 400 nm), while UVB filters are employed to block short-wave rays (295 to 320 m).

Oil-soluble UV filter substances include for example
3-benzylidenecamphor and its derivatives, e.g. 3-(4-methylbenzylidene)-camphor,
4-aminobenzoic acid derivatives, e.g. 4-(dimethylamino)benzoic acid-2-ethylhexyl ester, 4-(dimethylamino)benzoic acid-2-octyl ester or 4-(dimethylamino)benzoic acid amyl ester,
cinnamic acid esters, e.g. 4-methoxycinnamic acid-2-ethylhexyl ester, 4-methoxycinnamic acid isopentyl ester, 2-cyano-3-phenylcinnamic acid-2-ethylhexyl ester (octocrylenes),
salicylic acid esters, e.g. salicylic acid-2-ethylhexyl ester, salicylic acid-4-isopropylbenzyl ester, salicylic acid-homomentyl ester,
benzophenone derivatives, e.g. 2-hydroxy-4-methoxybenzophenone, 2-hydroxy-4-methoxy-4-methylbenzophenone, 2,2′-dihydroxy-4-methoxy-benzophenone,
benzalmalonic acid esters, e.g. 4-methoxybenalonic acid di-2-ethylhexyl ester,
triazine derivatives, e.g. 2,4,6-trianilino-(p-carbo-2′-ethyl-1′hexyloxy)-1,3,5-triazine, octyltriazone,
propane-1,3-diones, e.g. 1-(4-tert-butylphenyl)-3-(4′-methoxyphenyl)propane-1,3-dione.

Examples of water-soluble substances include
phenylbenzimidazolsulfonic acid and the salts thereof, e.g. alkali-, alkaline earth-, ammonium-, alkylammonium-, alkanolammonium- and glucammonium salts,
sulfonic acid derivatives of benzophenone, e.g. 2-hydroxy-4-methoxybenzophenone-5-sulfonic acid and the salts thereof,
sulfonic acid derivatives of 3-benzylidene camphor, e.g. 4-(2-oxo-3-bornylidenemethyl)benzenesulfonic acid and 2-methyl-5-(2-oxo-3-bornylidene)sulfonic acid and the salts thereof,
4-aminobenzoic acid derivatives, e.g. 4-(bis(polyethoxy)paraminobenzoic acid polyethoxyethyl ester.

Typical UV filters are especially octocrylene, 4-methoxycinnamic acid-2-ethylhexyl ester, 2-phenylbenzimidazol-5-sulfonic acid, 2-hydroxy-4-methoxybenzophenone sulfonic acid and 4-bis(polyethoxy)paraaminobenzoic acid polyethoxyethyl ester and the mixtures thereof.

In addition to the aforesaid soluble substances, insoluble pigments like finely dispersed metal oxides or salts are also suitable as UV filters, such as titanium dioxide, zinc oxide, iron oxide, aluminium oxide, cerium oxide, zirconium oxide, silicates (talcum), barium sulfate, and zinc stearate. The particles should have an average diameter of less than 100 nm, preferably from 5 to 50 nm, most preferably from 15 to 30 nm.

In addition to the abovementioned groups of primary sunscreens, secondary sunscreens may be employed as well.

Said substances may be chosen from among the group of antioxidants, which interrupt the photochemical chain reaction triggered by UV rays penetrating the skin. Typical examples include tocopherols and ascorbic acid and its esters.

Antidandruff Substances (E.2)

It has surprisingly been found that both water-soluble and oil-soluble antidandruff substances can readily be incorporated into the microemulsions.

Microemulsions containing antidandruff substances can favorably be employed as antidandruff shampoo for cleaning and treating the hair. In addition to reducing the dandruff formation, the oils in the microemulsion have a therapeutic effect on the scalp.

The term ‘antidandruff substance’ is used herein for substances which, besides their antiproliferative action, possess a ceratolytic effect. Said substances remove dandruff and microorganisms from the scalp. The therapeutic mechanism of antidandruff substances is the normalisation of the elevated cell division activity in the epidermis. The antidandruff substances furthermore have antimicrobial effects.

Examples of antidandruff substances include 1-hydroxy-4-methyl-6-(2,4,4-trimythylpentyl)-2-(1H)-pyridone-monoethanolamine salt, 1-acetyl-4-[4-[[2-(2,4-dichlorophenyl)-2-(1H-imidazol-1-ylmethyl)-1,3-dioxolane-4-yl]-methoxy]phenyl]piperazin, selenium disulfide, colloidal sulfur, sulfur polyethyleneglycolsorbitantonooleate, sulfur ricinoleic polyethoxylate, sulfur coal-tar distillates, salicylic acid (optionally combined with hexachlorophene), undecylenic acid monoethanolamide, sulfosuccinate sodium salt, potassium salt of the condensation product of undecylenic acid chloride and hydrolysed collagen, zinc pyrithione, aluminium pyrithione, and magnesium pyrithione/dipyrithione magnesium sulfate.

Most preferable oil-soluble antidandruff substances within the meaning of the invention are 1-(4-chlorophenoxy-1-(1H-imidazol-1-yl)-3,3-dimethyl-2-butanone (climbazol), and 3-aminopyridine (niacin amide).

A most preferable water-soluble antidandruff substance within the meaning of the invention is the compound comprised of 2-aminoethanol and 1-hydroxy-4-methyl-6-(2,4,4-trimethylpentyl)-2(1H)-pyridone (1:1) (piroctone olamine).

Other Surfactants or Emulsifiers (F)

In addition to the abovementioned alkanolammonium salts of the alkylsulfates and/or alkylpolyalkyleneglycolethersulfates, the microemulsions of the present invention may contain additional surfactants. Advantageously, one or more of these surfactants is (are) chosen from the group of
alcohol polyethyleneglycolethers, e.g. of the general formula R—O—(C₂H₄O)ₙ—H, where R is a branched or linear, saturated or unsaturated C₈- to C₂₀-alkyl residue and n is a number from 2 to 20; fatty acid ester polyethyleneglycolethers, e.g. of is the general formula
R—COO—(C₂H₄O)ₚ—H, where R is a branched or linear, saturated or unsaturated C₇- to C₁₉-alkyl residue and p is a number from 2 to 40,
alkyl polyalkyleneglycolethercarboxylic acids, e.g. of the general formula R—O—(C₂H₄O)ₙ—CH₂—COOH or the alkanol ammonium salts or alkali metal salts thereof, where R is a branched or linear, saturated or unsaturated C₈- to C₂₀-alkyl residue and n is a number from 2 to 20,
alkylamidoalkylbetains, e.g. of the general formula R—CONH(CH₂)ᵤN⁺(CH₃)₂—CH₂—COO⁻, where R is a branched or linear, saturated or unsaturated C₇- to C₁₉-alkyl residue and u is a number from 1 to 10,
products obtained by the alkoxylation of triglycerides, which are esterified, wholly or in part, with C₆- to C₂₂-fatty acids, wherein 2 to 40 moles of alkoxylation agent are employed per mole of triglyceride (as a most preferable surfactant group), e.g. addition products of castor oil and/or hydrogenated castor oil with ethylene oxide, which are partially esterified with oleic acid,
partially neutralised partial glycerides of mono- or polyvalent C₂- to C₂₂-carboxylic acids, such as linoleic acid, stearic acid, isostearic acid, palmitic acid, lauric acid, caprylic acid, capric acid, citric acid and/or lactic acid,
polyglycerol esters, wherein the carboxylic acid group preferably has 2 to 22 carbon atoms.

Preferably, the composition of the invention contains no or at most only small quantities (less than 0.5% by weight) of anionic surfactants of the sulfonate type.

Moreover, it is preferable that the composition of the invention contains only small amounts (less than 0.5% by weight) of fatty acid polyglycol ester sulfates. Most preferably, the composition particularly does not contain any fatty acid polyglycol ester sulfates.

Electrolytes (G)

The microemulsions of the present invention may contain electrolytes. Examples thereof include alkali salts and alkaline earth salts, such as the corresponding halides, sulfates, phosphates, or citrates.

Additives (H)

Examples of additives include poly(C₂- to C₄-)alkyleneglycols, particularly polyethylene glycols and/or polypropylene glycols, each preferably with a molecular weight of up to 1,500 g/mole, fragrances, colorants, hydrotropes, thickeners, pearlescent agents, protein hydrolysates, plant extracts, vitamins, α-hydroxycarboxylic acids and their esters, antimicrobials and the like. According to the present invention, UV filters (cf. E.1) and antidandruff substances (cf. E.2) are not defined as additives (cf. H),

The compositions of the invention are especially appropriate for producing foams which can be applied through a manually operated foam dispensing pump without the need for a propellant, e.g. the products commercialized by Airspray International.

The following examples are merely illustrative and are not intended to constitute a limitation on the present invention. The term ‘percent’ shall mean ‘percent by weight’, based on the total weight of the respective microemulsion.

### EXAMPLE 1

| | |
|---|---|
| a) MARLINAT ™ 242/90 M | 15.5% |
| MARLOWET ™ LVS | 3.9% |
| Avocado oil | 0.7% |
| Jojoba oil | 0.2% |
| MARLINAT ™ CM 105/80 | 1.9% |
| b) Neo Heliopan ™ Hydro* | 4.0% |
| Deionized water | 63.6% |
| NaOH, 20% | 2.6% |
| NaCl | 1.9% |
| Ampholyt JB 130 K | 2.7% |
| c) Antil ™ 141, liquid | 1.0% |
| Lauryl glucoside | 1.0% |
| Perfume | 1.0% |
| Preservative | q.s. |

*adjusted at pH 6.5 with 20% NaOH solution

Preparation

The temperature for the whole procedure was 20° C.

The components listed under a) were combined one after the other and were stirred to give a homogeneous blend. The same was done with the components listed under b). Blend b) then was slowly added to blend a) with continuous stirring. Finally, the components listed under c) were added to the combined clear blend with continuous stirring. The resultant product had a high foaming power.

### EXAMPLE 2

| | |
|---|---|
| a) MARLINAT ™ 242/90 M | 16.0% |
| MARLOWET ™ LVS | 4.0% |
| Avocado oil | 0.6% |
| Jojoba oil | 0.2% |
| MARLINAT ™ CM 105/80 | 2.0% |
| b) Deionized water | 65.4% |
| NaCl | 3.0% |
| Ampholyt JB 130 K | 2.8% |
| c) Antil ™ 141, liquid | 3.0% |
| Perfume | 1.0% |
| Preservative | q.s. |

Preparation: As described in Example 1.

### EXAMPLE 3

| | |
|---|---|
| a) MARLINAT ™ 242/90 M | 28.0% |
| MARLOWET ™ LVS | 7.0% |
| LIPOXOL ™ 600 | 2.0% |
| Soybean oil | 4.0% |
| Castor oil | 1.0% |
| MARLINAT ™ CM 105/80 | 3.8% |
| Protein hydrolysate | 0.5% |
| Uvinul ™ MC 80 | 3.0% |
| b) Deionized water | 38.7% |
| NaCl | 2.0% |
| Ampholyt JB 130 K | 5.0% |
| Uvinul ™ MS 40* | 2.0% |
| c) Antil ™ 141, liquid | 2.0% |
| Perfume | 1.0% |
| Preservative | q.s. |

*adjusted at pH 6.5 with 20% NaOH solution

Preparation: As described in Example 1.

### EXAMPLE 4

| | |
|---|---|
| a) MARLINAT ™ 242/90 M | 28.0% |
| MARLOWET ™ LVS | 7.0% |
| LIPOXOL ™ 600 | 2.0% |
| Soybean oil | 4.0% |
| Castor oil | 1.0% |
| MARLINAT ™ CM 105/80 | 3.8% |
| Protein hydrolysate | 0.5% |
| Uvinul ™ MC 80 | 3.0% |
| b) Deionized water | 39.7% |
| NaCl | 1.0% |
| Ampholyt JB 130 K | 5.0% |
| Uvinul ™ P 25 | 2.0% |
| c) Antil ™ 141, liquid | 2.0% |
| Perfume | 1.0% |
| Preservative | q.s. |

Preparation: As described in Example 1.

### EXAMPLE 5

| | |
|---|---|
| a) MARLINAT ™ 242/90 M | 19.0% |
| MARLOWET ™ LVS | 2.5% |
| RonaCare ™ Nicotinamide | 0.7% |
| Jojoba oil | 1.0% |
| MARLINAT ™ CM 105/80 | 2.3% |
| b) Deionized water | 68.6% |
| NaCl | 2.4% |
| Ampholyt JB 130 K | 1.8% |
| c) Antil ™ 141, liquid | 0.6% |
| Perfume | 0.6% |
| Preservative | q.s. |

Preparation: As described in Example 1.

### EXAMPLE 6

| | |
|---|---|
| a) MARLINAT ™ 242/90 M | 15.0% |
| MARLOWET ™ LVS | 2.0% |
| Crinipan ™ AD | 0.7% |
| Jojoba oil | 1.0% |
| MARLINAT ™ CM 105/80 | 1.8% |
| b) Deionized water | 74.0% |
| NaCl | 2.0% |
| Ampholyt K JB 130 | 1.5% |
| c) Antil ™ 141, liquid | 1.0% |
| Perfume | 0.5% |
| Preservative | q.s. |

Preparation: As described in Example 1.

### EXAMPLE 7

| | |
|---|---|
| a) MARLINAT ™ 242/90 M | 19.0% |
| MARLOWET ™ LVS | 2.5% |
| Octopirox ™ | 0.5% |
| Jojoba oil | 1.0% |
| MARLINAT ™ CM 105/80 | 2.3% |
| b) Deionized water | 67.3% |
| NaCl | 2.4% |
| Ampholyt JB 130 K | 1.8% |
| c) Antil ™ 141, liquid | 0.6% |
| Aloe vera (1:1) | 2.0% |
| Perfume | 0.6% |
| Preservative | q.s. |

Preparation: As described in Example 1.

The following products were used in Examples 1 to 7:

| | |
|---|---|
| MARLINAT ™ | C₁₂/C₁₄-alkylpolyethyleneglycol(2 EO)ether- |
| 242/90 M | sulfate-monoisopropanolammonium salt in 1,2- |
| | propyleneglycol (Sasol Germany GmbH) |
| MARLINAT ™ | C₁₂/C₁₄-alkylpolyethyleneglycol(10 |
| CM 105/80 | EO)ethercarboxylic acid sodium salt (Sasol |
| | Germany GmbH) |
| MARLOWET ™ LVS | Ethoxylated castor oil, partially esterified with |
| | oleic acid |
| | (Sasol Germany GmbH) |
| LIPOXOL ® 600 | Polyethyleneglycol 600 |
| | (Sasol Germany GmbH) |
| Ampholyt JB 130 K | Cocoamidopropyldimethylbetaine |
| | (Sasol Germany GmbH) |
| Neo Heliopan ™ | 2-Phenylbenzimidazol-5-sulfonic acid |
| Hydro | (Haarmann & Reimer GmbH) |
| Neo Heliopan ™ 303 | Octocrylenes |
| | (Haarmann & Reimer GmbH) |
| Antil ™ 141, liquid | Polyethyleneglycol-propyleneglycol-dioleate |
| | (Degussa AG) |
| Uvinul ™ MC 80 | 4-Methoxycinnamic acid-2-ethylhexyl ester |
| | (BASF AG) |
| Uvinul ™ MS 40 | 2-Hydroxy-4-methoxybenzophenone-5-sulfonic |
| | acid (BASF AG) |
| Uvinul ™ P 25 | 4-Bis(polyethoxy)paraminobenzoic acid |
| | polyethoxyethyl ester |
| | (BASF AG) |
| RonaCare ™ | Niacin amide (Merck KGaA) |
| Nicotinamide | |
| Crinipan ™ AD | Climbazol (Haarmann & Reimer GmbH) |
| Octopyrox ™ | Piroctone olamine (Clariant GmbH) |

The formulations given herein as examples are outstanding in their high cleaning and foaming power, good initial foaming power, storage stability, and mildness to the skin.

1. A microemulsion comprising at least the following components:
(A) 0.5 to 70% by weight of alkanolammonium salts of the alkylsulfates and/or alkylpolyalkyleneglycolethersulfates having the following structure
R¹—O—(CₚH₂ₚO)ₘ—SO₃—HN⁺R²R³R⁴,
wherein
R¹=is a C₈- to C₂₀-hydrocarbon radical,
p=is an integer from 2 to 5, where p can be different for each m,
R²=is H, a C₁- to C₆-alkyl or a C₂- to C₄-hydroxyalkyl,
R³=is H, a C₁- to C₆-alkyl or a C₂- to C₄-hydroxyalkyl,
R⁴=is a C₂- to C₄-hydroxyalkyl, and
m=is an integer from 0 to 7,
or mixtures thereof,
(B) 20 to 95% by weight of water,
(C) 0.1 to 20% by weight of one or more oil component(s), and
(D) 0.1 to 20% by weight of one or more mono- or polyhydroxy C₂- to C₂₄-alcohol(s), and
an additive selected from the group consisting of:
(E.1) 0.1 to 15% by weight of one or more UV filter(s)
(E.2) 0.1 to 3% by weight of one or more antidandruff substance(s) and mixtures thereof, each percentage hereof based on the total composition.

2. A microemulsion according to claim 1,
characterized in that the UV filter(s) is (are) chosen from among the group of 3-benzylidenecamphor and its derivatives, 4-aminobenzoic acid derivatives, cinnamic acid esters, salicylic acid esters, benzophenone derivatives, benzalmalonic acid esters, triazine derivatives, propane-1,3-diones, phenylbenzimidazolsulfonic acid and the salts thereof, sulfonic acid derivatives of benzophenone, sulfonic acid derivatives of 3-benzylidene camphor, 4-aminobenzoic acid derivatives, and finely dispersed metal oxides or salts.

3. A microemlusion according to any one of the preceding claims,
characterized in that the UV filter (E.1) is one or more of the following substances: octocrylenes, 4-methoxycinnamic acid-2-ethylhexyl ester, 2-phenylbenzimidazol-5-sulfonic acid, 2-hydroxy-4-methoxybenzophenone sulfonic acid, and 4-bis(polyethoxy)paraminobenzoic acid polyethoxyethyl ester, and mixtures thereof.

4. A microemulsion according to claim 1,
characterized in that the antidandruff substance (E.2) is one or more of the following substances: 1-(4-chlorophenoxy-1-(1-H-imidazol-1-yl)-3,3-di-methyl-2-butanone, 3-aminopyridine, and the compound composed of 2-aminoethanol and 1-hydroxy-4-methyl-6-(2,4,4-trimethylpentyl)-2(1H)-pyridone.

5. A microemulsion according to claim 1,
characterized in that the alkanolammonium salts of the alkylsulfates and/or alkylpolyalkyleneglycolethersulfates have independently of one another the following residues or indices:
R¹=a linear or saturated C₁₂- to C₁₆-alkyl residue,
p=2 or 3, where p may be different for each m,
R²=H or hydroxyisopropyl,
R³=H or hydroxyisopropyl,
R⁴=hydroxyisopropyl and/or
m=an integer from 0 to 2.

6. A microemulsion according to any one of claims 1 or 2, wherein the microemulsion contains
2 to 60% by weight of component (A)
30 to 80% by weight of component (B)
0.5 to 15% by weight of component (C) and/or
0.1 to 9% by weight of component (D).

7. A microemulsion according to any one of claims 1 or 2, wherein the microemulsion also contains at least one of the following components:
(F) greater than 0 to 20% by weight of one or more additional surfactant(s) or emulsifier(s),
(G) greater than 0 to 20% by weight of one or more electrolyte(s), and
(H) greater than 0 to 10% by weight of one or more additive(s),
and optionally also

8. A microemulsion according to claim 7 containing at least the following component:
(F) at least 1% by weight of a product obtained by the alkoxylation of triglycerides, which is esterified, wholly or in part, with C₆- to C₂₂-fatty acids, wherein preferably 2 to 40 moles of alkoxylation agent are employed per mole of triglyceride.

9. A microemulsion according to any one of claims 1 or 2, wherein the oil component (C) contains one or more component(s) chosen from the group of lecithins, mono-, di-, and/or triglycerides of saturated and/or unsaturated, branched and/or linear carboxylic acids having a chain length from 8 to 24 carbon atoms, branched and/or linear hydrocarbons, waxes, Vaseline, paraffin oils, polyolefins, silicone oils, and esters of saturated, unsaturated and/or aromatic, branched and/or linear carboxylic acids having a chain length from 3 to 30 carbon atoms and saturated and/or unsaturated, branched and/or linear alcohols having a chain length from 3 to 30 carbon atoms, and mixtures thereof.

10. A microemulsion according to any one of claims 1 or 2, characterized in that the microemulsion is a stable and transparent emulsion with an average particle size of less than 100 nm in its disperse phase.

11. A microemulsion according to any one of claims 1 or 2, characterized in that the microemulsion contains less than 0.5% by weight of anionic surfactants of the sulfonate type and particularly less than 0.5% by weight of fatty acid polyglycol ester sulfates, preferably no fatty acid polyglycol ester sulfates.

12. The use of the microemulsion according to any one of claims 1 or 2 in cosmetic applications and/or the production of a medical product for medicinal-dermatologic applications.

13-17. (canceled)

18. A method of protecting skin by applying to the skin a microemulsion according to any one of claims 1 or 2, wherein the emulsion contains the component (E.1) as a sunscreen.

19. A method for cleaning and treating the skin by applying the microemulsion of any one of claims 1 or 2 in the form of a foam generated by means of a manually operated pump for dispensing foam without using propellants.

20. The method of claim 18 wherein the microemulsion contains more than 0% by weight of the component (F) and can be rinsed off with water after application.

21. The method claim 19 wherein the microemulsion contains more than 0% by weight of the component (F) and can be rinsed off with water after application.

22. A method of cleaning and treating hair, particularly as a shower gel, which can be rinsed off with water after application, comprising applying to hair the microemulsion of any one of claims 1 or 2.

23. A method of cleaning hair comprising applying to hair the microemulsion according to any one of claims 1 or 2 as an antidandruff shampoo, which contains the component (E.2) and preferably also contains an amount of component (F).

24. A method according to claim 23, wherein component (E.2) is 3-aminopyridine (niacin amide) and/or 1-(4-chlorophenoxy-1-(1H-imidazol-1-yl)-3,3-dimethyl-2-butanone (climbazol).

25. A microemulsion according to claim 4, characterized in that component (E.2) is 3-aminopyridine (niacin amide).

26. A microemulsion according to claim 4, characterized in that component (E.2) is 1-(4-chlorophenoxy-1-(1H-imidazol-1-yl)-3,3-dimethyl-2-butanone (climbazol).
